SEQUENCE LISTING (2 sequences)

SEQ ID NO: 1 - DNA, 1125 bp, Homo sapiens
ttctgccctcgagcccaccgggaacgaaagagaagctctatctcgcctccaggagcccagctatgaactccttctccacaagcgccttcggtccagttgccttctccctggggctgctcctggtgttgcctgctgccttccctgccccagtacccccaggagaagattccaaagatgtagccgccccacacagacagccactcacctcttcagaacgaattgacaaacaaattcggtacatcctcgacggcatctcagccctgagaaaggagacatgtaacaagagtaacatgtgtgaaagcagcaaagaggcactggcagaaaacaacctgaaccttccaaagatggctgaaaaagatggatgcttccaatctggattcaatgaggagacttgcctggtgaaaatcatcactggtcttttggagtttgaggtatacctagagtacctccagaacagatttgagagtagtgaggaacaagccagagctgtgcagatgagtacaaaagtcctgatccagttcctgcagaaaaaggcaaagaatctagatgcaataaccacccctgacccaaccacaaatgccagcctgctgacgaagctgcaggcacagaaccagtggctgcaggacatgacaactcatctcattctgcgcagctttaaggagttcctgcagtccagcctgagggctcttcggcaaatgtagcatgggcacctcagattgttgttgttaatgggcattccttcttctggtcagaaacctgtccactgggcacagaacttatgttgttctctatggagaactaaaagtatgagcgttaggacactattttaattatttttaatttattaatatttaaatatgtgaagctgagttaatttatgtaagtcatatttatatttttaagaagtaccacttgaaacattttatgtattagttttgaaataataatggaaagtggctatgcagtttgaatatcctttgtttcagagccagatcatttcttggaaagtgtaggcttacctcaaataaatggctaacttatacatatttttaaagaaatatttatattgtatttatataatgtataaatggtttttataccaataaatggcattttaaaaaattc

SEQ ID NO: 2 - DNA, 3106 bp, Homo sapiens
gcggccgcgctccggccgctttgtcgaaagccggcccgactggagcaggacgaagggggagggtctcgaggccgagtcctgttcttctgagggacggaccccagctggggtggaaaagcagtaccagagagcctccgaggcgcgcggtgccaaccatggagcgggccggccccagcttcgggcagcagcgacagcagcagcagccccagcagcagaagcagcagcagagggatcaggactcggtcgaagcatggctggacgatcactgggactttaccttctcatactttgttagaaaagccaccagagaaatggtcaatgcatggtttgctgagagagttcacaccatccctgtgtgcaaggaaggtatcagaggccacaccgaatcttgctcttgtcccttgcagcagagtcctcgtgcagataacagtgtccctggaacaccaaccaggaaaatctctgcctctgaatttgaccggcctcttagacccattgttgtcaaggattctgagggaactgtgagcttcctctctgactcagaaaagaaggaacagatgcctctaacccctccaaggtttgatcatgatgaaggggaccagtgctcaagactcttggaattagtgaaggatatttctagtcatttggatgtcacagccttatgtcacaaaattttcttgcatatccatggactgatatctgctgaccgctattccctgttccttgtctgtgaagacagctccaatgacaagtttcttatcagccgcctctttgatgttgctgaaggttcaacactggaagaagtttcaaataactgtatccgcttagaatggaacaaaggcattgtgggacatgtggcagcgcttggtgagcccttgaacatcaaagatgcatatgaggatcctcggttcaatgcagaagttgaccaaattacaggctacaagacacaaagcattctttgtatgccaattaagaatcatagggaagaggttgttggtgtagcccaggccatcaacaagaaatcaggaaacggtgggacatttactgaaaaagatgaaaaggactttgctgcttatttggcattttgtggtattgttcttcataatgctcagctctatgagacttcactgctggagaacaagagaaatcaggtgctgcttgaccttgctagtttaatttttgaagaacaacaatcattagaagtaattttgaagaaaatagctgccactattatctctttcatgcaagtgcagaaatgcaccattttcatagtggatgaagattgctccgattctttttctagtgtgtttcacatggagtgtgaggaattagaaaaatcatctgatacattaacaagggaacatgatgcaaacaaaatcaattacatgtatgctcagtatgtcaaaaatactatggaaccacttaatatcccagatgtcagtaaggataaaagatttccctggacaactgaaaatacaggaaatgtaaaccagcagtgcattagaagtttgctttgtacacctataaaaaatggaaagaagaataaagttataggggtttgccaacttgttaataagatggaggagaatactggcaaggttaagcctttcaaccgaaatgacgaacagtttctggaagcttttgtcatcttttgtggcttggggatccagaacacgcagatgtatgaagcagtggagagagccatggccaagcaaatggtcacattggaggttctgtcgtatcatgcttcagcagcagaggaagaaacaagagagctacagtcgttagcggctgctgtggtgccatctgcccagacccttaaaattactgactttagcttcagtgactttgagctgtctgatctggaaacagcactgtgtacaattcggatgtttactgacctcaaccttgtgcagaacttccagatgaaacatgaggttctttgcagatggattttaagtgttaagaagaattatcggaagaatgttgcctatcataattggagacatgcctttaatacagctcagtgcatgtttgctgctctaaaagcaggcaaaattcagaacaagctgactgacctggagatacttgcattgctgattgctgcactaagccacgatttggatcaccgtggtgtgaataactcttacatacagcgaagtgaacatccacttgcccagctttactgccattcaatcatggaacaccatcattttgaccagtgcctgatgattcttaatagtccaggcaatcagattctcagtggcctctccattgaagaatataagaccacgttgaaaataatcaagcaagctattttagctacagacctagcactgtacattaagaggcgaggagaattttttgaacttataagaaaaaatcaattcaatttggaagatcctcatcaaaaggagttgtttttggcaatgctgatgacagcttgtgatctttctgcaattacaaaaccctggcctattcaacaacggatagcagaacttgtagcaactgaattttttgatcaaggagacagagagagaaaagaactcaacatagaacccactgatctaatgaacagggagaagaaaaacaaaatcccaagtatgcaagttgggttcatagatgccatctgcttgcaactgtatgaggccctgacccacgtgtcagaggactgtttccctttgctagatggctgcagaaagaacaggcagaaatggcaggcccttgcagaacagcaggagaagatgctgattaatggggaaagcggccaggccaagcggaactgagtggcctatttcatgcagagttgaagtttacagagatggtgtgttctgcaatatgcctagtttcttacacactgtctgtatagtgtctgtatttggtatatactttgccactgctgtatttttatttttgcacaacttttgagagtatagcatgaatgtttttagaggactattacatattttttgtatatttgttttatgctactgaactgaaaggatcaacaacatccactgttagcacattgataaaagcattgtttgtgatatttcgtgtactgcaaagtgtatgcagtattcttgcactgaggtttttttgcttgggg

(19) United States
(12) Patent Application Publication
(10) Pub. No.: US 2009/0176283 A1
(43) Pub. Date: Jul. 9, 2009

(54) METHOD FOR THE OBTENTION OF CHIMERIC NUCLEOTIDE SEQUENCES AND CHIMERIC NUCLEOTIDE SEQUENCES

(76) Inventors: Diogenes Santiago Santos, Porto Alegre (BR); Luiz Augusto Basso, Porlo Alegre (BR); Jocelei Maria Chies, Porto Alegre (BR); Isabel Osorio da Fonseca, Pelotas (BR); Gaby Renard, Porto Alegre (BR)

Correspondence Address: SMITH, GAMBRELL & RUSSELL, SUITE 3100, PROMENADE II, 1230 PEACHTREE STREET, N.E., ATLANTA, GA, 30309-3592, US

(21) Appl. No.: 12/096,267

(22) PCT Filed: Dec. 8, 2006
(86) PCT No.: PCT/BR2006/000267

§ 371 Date: Sep. 23, 2008

(30) Foreign Application Priority Data
Dec. 12, 2005 (BR) PI0506047.8

(51) Int. Cl. C12P 19/34

(52) U.S. Cl. 435/91.4; 435/91.1

(57) ABSTRACT

The present invention describes a method for producing synthetic nucleotide sequences which provides the assembly of DNA sequences, thus providing the obtention of genes, chromosomes and even whole qenomes. The method of the present invention makes use of the technique known as Polymerase Chain Reaction (PCR) but wherein no preexisting nucleic acid template is needed, being therefore an approach with minimum limitations and broad use. This method provides means for obtaining products with high industrial value, for the design and development of immunotherapeutic agents, recombinant enzymes, drugs, including the development of vaccines, gene therapy, and in applications in agriculture and environment.

### STATEMENT OF RELATED APPLICATIONS

This application is the U.S. National Phase Under Chapter II of the Patent Cooperation Treaty (PCT) of PCT International Application No. PCT/BR2006/000267 having an International Filing Date of 8 Dec. 2006, which claims priority on Brazilian Patent Application No. PI0506047-8 having a filing date of 12 Dec. 2005.

### BACKGROUND OF THE INVENTION

1. Technical Field

The present invention describes a method for producing synthetic nucleotide sequences. More specifically, the method provides the assembly of DNA sequences, thus providing the obtention of genes, chromosomes and even whole genomes. This method provides means for obtaining products with high industrial value, for the design and development of immunotherapeutic agents, recombinant enzymes, drugs, including the development of vaccines, gene therapy, and in applications in agriculture and environment. The method of the present invention makes use of the technique known as Polymerase Chain Reaction (PCR) but wherein no preexisting nucleic acid template is needed, being therefore an approach with minimum limitations and broad use.

2. Related Art

Recombinant DNA technology is a powerful technology, although in most cases being so far limited by requiring preexisting DNA sequences as starting points. Such sequences can be altered through the use of restriction enzymes, initiator oligonucleotides (also referred to in the art as primers) for DNA amplification, site specific mutagenesis and other techniques. Current recombinant DNA technologies do not satisfactory enable the creation of molecules, genes, genomes or completely artificial organisms. Only genetic modifications of natural organisms are enabled by the current techniques, which can be useful in biotechnological or commercial efforts to design and develop recombinant enzymes with therapeutic applications, including immunotherapeutic agents, development of vaccines, gene therapy, and in applications in agriculture and environment. However, current technology depends on organisms and or DNA molecules already present/available in nature.

To improve or create new functions to nucleotide sequences, or to modify organisms for specialized uses such as, for example, the production of hormones, laborious manipulation is usually necessary, thus consuming time-, human-, and financial resources. Some modifications on naturally occurring DNA can be so complex that are almost impossible to be performed in practice. Therefore, there exists a need for alternative approaches to the currently available technologies, so as to enable the creation of new nucleotide sequences without the need of using pre-existing DNA templates such as naturally occurring ones and/or those obtained by recombinant DNA technology.

There are two well-known general methods for the synthetic assembly of oligonucleotides in long fragments, also referred to as polynucleotides:
1) The first method is the oligonucleotides assembly that covers the entire sequence that is being synthesized. In this method, the first step comprises annealing and DNA ligase fulfilling of the spaces. Afterwards the fragment is directly cloned or cloned after the amplification by PCR. The polinucleotides are subsequently used in vitro to assemble the complex in a longer sequence.
2) The second method refers to gene synthesis, using DNA polymerase to fulfill the single strand spaces between the homologous regions of paired nucleotides in a single step. After reaction with DNA polymerase, the single strand regions become double stranded and, after digestion with restriction endonucleases, they can be directly cloned or used to obtain one or more complexes of additional sequences by different bonding of double stranded fragments. In this method there are several possibilities in the order of oligonucleotides bonding, demanding the sequencing of the generated clones to confirm the obtention of the intended recombinant DNA.

The state of the art comprises some documents only partially related to the subject-matter of the present invention, and no particularly relevant document was found in this regard. The international patent application WO 04/113534, filed by University of California and entitled “Method for producing a synthetic gene or other DNA sequence”, describes a method to synthesize nucleic acid sequences. Said method comprises the steps of: dividing the desired sequence into a plurality of partially overlapping segments; and optimizing the melting temperatures of the overlapping regions of each segment to disable hybridization of the non-adjacent overlapping segments in the desired sequence. This strategy allows the choice of conditions that favor the hybridization of overlapping regions of single stranded segments which are adjacent to a hybridized sequence and disfavor the hybridization of non-adjacent segments; and filling in, bonding, or repairing the gaps between the overlapping regions, thereby forming a double stranded DNA with the desired sequence.

The European Patent EP 1 392 868, filed by Wisconsin Alumni Res. Found. (US) and entitled “Method for the synthesis of DNA sequences”, describes a method for the direct synthesis of double stranded DNA molecules of variable sizes and with any desired sequence. The DNA molecule to be synthesized is broken into smaller overlapping DNA segments. A maskless microarray synthesizer is used to make a DNA microarray on a substrate in which each element or feature of the array is populated by DNA of one of the overlapping DNA segments. The DNA segments are released from the substrate and held under conditions favoring hybridization of DNA, and under such conditions the segments will spontaneously hybridize together to form the desired DNA construct.

The European Patent EP 1 538 206, assigned to Egea Biosciences LLC and entitled “Method for the complete chemical synthesis and assembly of genes and genomes”, describes another method concerning the synthesis and assembly of DNA fragments. Said method can give rise to completely artificial genes, chromosomes and even genomes, but uses a different approach, using known sequences or sequences available in databases. This method uses computer programs to perform the combinatorial assembly of oligonucleotides and series of steps that use other enzymes to obtain the intended products.

The present invention aims to overcome several unsolved problems of the current related techniques, including: i) to provide a method for enabling the creation of molecules, genes, whole genomes, and/or completely artificial organisms, said method not being limited to modifying naturally occurring organisms; ii) to provide a method that enables the creation of new nucleotide sequences without the need of using as template either naturally occurring DNA molecules or preexisting recombinant DNA.

### BRIEF SUMMARY OF THE INVENTION

The present invention has as one of its objectives to provide a method for obtaining nucleotide sequences without requiring preexisting template DNA. The method of the invention is based on the design of at least two initiator oligonucleotides corresponding to the target double stranded DNA, in a continuous, successive and alternate method. The method of the invention and its assembly step(s) uses just one enzyme, as a thermostable DNA polymerase and avoids the screening of several clones to obtain the intended sequence.

The synthesized target sequence is linked to the vector to obtain the desired chimerical DNA.

The proposed method circumvents the limitations of current DNA manipulation techniques. The method of the invention provides a faster and more efficient means to obtain any artificial DNA sequence with high efficiency and precision. The following figures are part of the present invention and are intended to illustrate some of its preferred embodiments but shall not limit its scope. The method of the invention will be better understood if the reader follows the detailed description along with the figures

### BRIEF DESCRIPTION OF THE DRAWING FIGURES

FIG. 1 shows a diagram for synthetic DNA sequence synthesis (genes, chromosomes and or genomes) which code a target product.

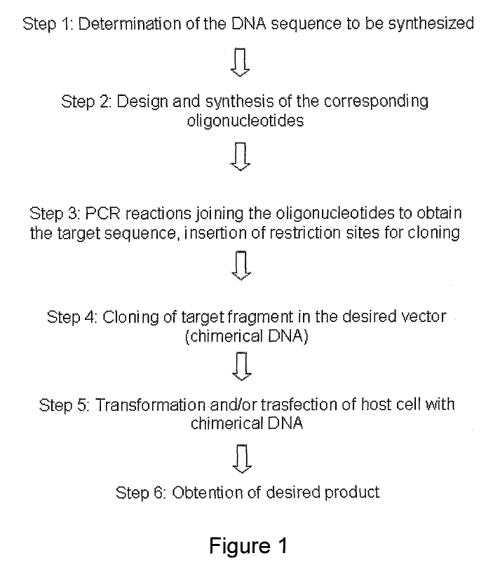

FIG. 2 shows a DNA sequence of the gene coding for the human Interferon β2, used as an example for the synthesis of a 650 base pairs sequence. The length of oligonucleotides used to construct the profit region of the gene is indicated in the continuous lines. The upper line shows the region used for designing the initiator oligonucleotides in the upper strip. The bottom line indicates the region used to design the initiator oligonucleotides in the bottom strip (opposite).

FIG. 3 shows the schematics of amplification and assembly of a desired DNA sequence of up to 650 base pairs (bp). On (a) the oligonucleotides used are schematized. The first step (b) shows the overlap of 3′ end of P1 with the 3′ end of P2 and extension of these oligonucleotides. The same procedure is followed, on separated reactions, for P3 and P4, P5 and P6, P7 and P8, P9 and P10, P11 and P12, and P13 and P14. On (c) the obtained products from (b), purified from agarose gel, are joined (P1+P2 with P3+P4) and amplified in the presence of external oligonucleotides P1 and P4. The same procedure is performed with P5+P6 and P7+P8 and for P9+P10 and P11+P12, in the presence of P5 and P8, and P9 and P12 oligonucleotides, respectively. The obtained fragments are purified from agarose gel. In the third step (d), the P1+P2+P3+P4 fragment is joined to P5+P6+P7+P8 fragment and amplified in the presence of external oligonucleotides P1 and P8. The P9+P10+P11+P12 fragment is joined to P13+P14 in the presence of external oligonucleotides P9 and P14. In the fourth and last step (e), P1+P2+P3+P4+P5+P6+P7+P8 fragment is joined to P9+P10+P11+P12+P13+P14 fragment and amplified in presence of external oligonucleotides P1 and P14. On (f) the product that corresponds to target DNA sequence (gene of β-2 human interferon) is exemplified and extended.

FIG. 4 shows a DNA sequence of the gene coding the human phosphodiesterase 5A enzyme, used as an example of synthesis of a 970 base pairs sequence. The length of initiator oligonucleotides used to construct the profit region of the gene (catalytic domain) is indicated by the continuous lines. The upper line shows the region used for designing the initiator oligonucleotides in the upper strip. The bottom line indicates the region used for designing the initiator oligonucleotides in the bottom strip (opposite).

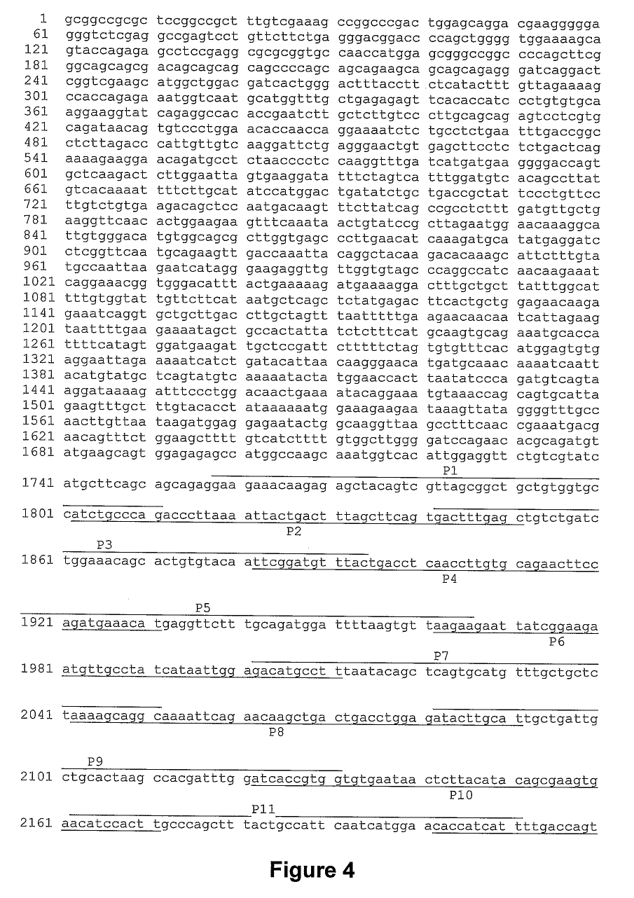

FIG. 5 shows the schematics of amplification and assembly of desired DNA sequence of 970 base pairs. On (a) the initiator oligonucleotides used are represented. The first step (b) shows the overlap of 3′ end of P1 with the 3′ end of P2 and extension of these oligonucleotides. The same procedure is followed, on separated reactions, for P3 and P4, P5 and P6, P7 and P8, P9 and P10, P11 and P12, P13 and P14, P15 and P16, P17 and P18, P19 and P20, P21 and P22, and P23 and P24. On (c) the obtained products on (b), purified from agarose gel, are joined (P1+P2 with P3+P4) and amplified in the presence of external oligonucleotides P1 and P4. The same procedure is performed for P5+P6 and P7+P8 in presence of P5 and P8, for P9+P10 and P11+P12 in presence of P9 and P12, for P13+P14 and P15+P16 in presence of P13 and P16, for P17+P18 and P19+P20 in presence of P17 and P20 and for P21+P22 and P23+P24 in presence of P21 and P24. The obtained fragments are purified from agarose gel. To the third step (d) P1+P2+P3+P4 fragment is joined to P5+P6+P7+P8 fragment and amplified in the presence of external oligonucleotides P1 e P8. The P9+P10+P11+P12 fragment is joined to P13+P14+P15+P16 fragment in presence of external oligonucleotides P9 and P16. The P17+P18+P19+P20 fragment is joined to P21+P22+P23+P24 fragment in presence of external oligonucleotides P17 and P24. The obtained fragments are purified from agarose gel. The step (e) is used to increase the homology region among the products. For achieving this, the P1+P2+P3+P4+P5+P6+P7+P8 fragment is joined to P9+P10+P11+P12 fragment and amplified in the presence of external oligonucleotides P1 and P12. The P13+P14+P15+P16 fragment is joined to P17+P18+P19+P20+P21+P22+P23+P24 fragment in presence of external oligonucleotides P17 and P24. In the intermediary step, the P1+P2+P3+P4+P5+P6+P7+P8+P9+P10+P11+P12 product is joined to P9+P10+P11+P12+P13+P14+P15+P16 product in presence of P1 and P16 and the P9+P10+P11+P12+P13+P14+P15+P16 product is also joined, in a separated reaction, to P13+P14+P15+P16+P17+P18+P19+P20+P21+P22+P23+P24 product in presence of P13 and P24. In step (f), the P1+P2+P3+P4+P5+P6+P7+P8+P9+P10+P11+P12 product is joined to P9+P10+P11+P12+P13+P14+P15+P16+P17+P18+P19+P20+P21+P22+P23+P24 product, in presence of P1 e P24 oligonucleotides, to obtain the final desired product (g).

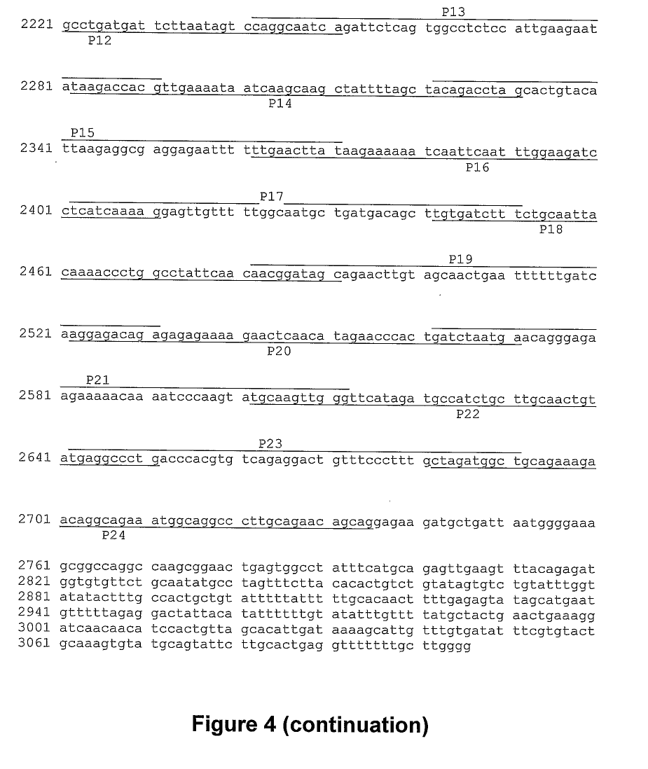

FIG. 6 shows the amplified products in an agarose gel, in each step of the process described in FIG. 3 for assembling the sequence that code the catalytic domain from phosphodiesterase 5A enzyme. Agarose gel 2% stained with ethidium bromide. In A, lane 1, the product of the first step of amplification (e.g., P1+P2) is shown, where the product is 90 bp. In B, lane 2, the product of the second stage of amplification is shown (e.g.; joining of P1+P2 with P3+P4), in which the resulting product has about 170 bp. In C, lane 3, the product of the third step of amplification is shown (e.g.; joining of P1+P2+P3+P4 with P5+P6+P7+P8) that should result in a 330 bp amplification product. In D, lane 4, the product of the fourth step of amplification is shown (e.g.; joining P1+P2+P3+P4+P5+P6+P7+P8 with P9+P10+P11+P12) that shows an amplification product of about 500 bp. In the last step E, lane 5, the 970 bp final amplification product was obtained, indicated by the asterisk, (P1+P2+P3+P4+P5+P6+P7+P8+P9+P10+P1+P12+P13+P14+P15+P16+P17+P18+P19+P20+P21+P22+P23+P24). All products were purified from agarose gel to be used in subsequent steps. M-DNA molecular weight markers (1 kb Plus).

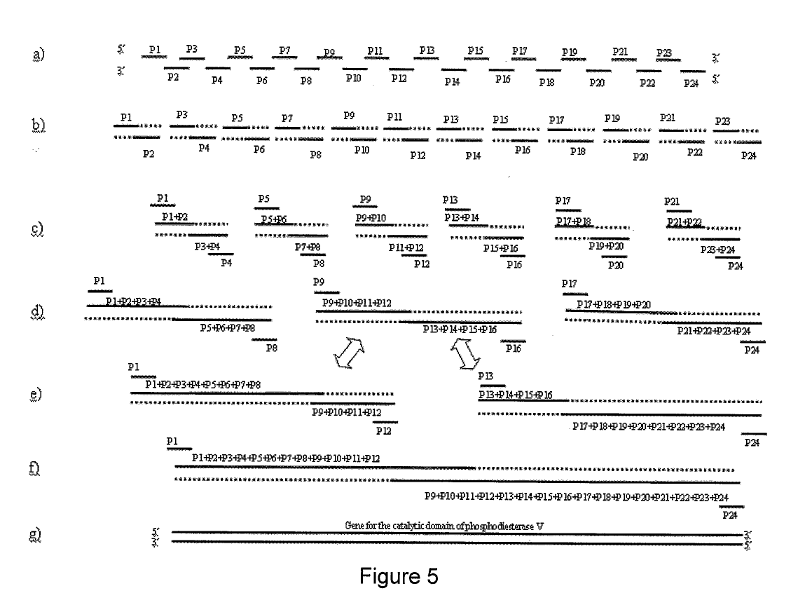

FIG. 7 shows an example of the efficiency confirmation of the method of the invention, where the protein corresponding to the catalytic domain of human 5A phosphodiesterase coded by DNA sequence, obtained by said method was duly expressed in Escherichia coli. The figure shows a SDS-PAGE gel where the indicated band (1) corresponds to the catalytic domain of human 5A phosphodiesterase as produced by Escherichia coli K12 hosting a pBR322 vector. 1—overexpression of desired protein; 2—control.

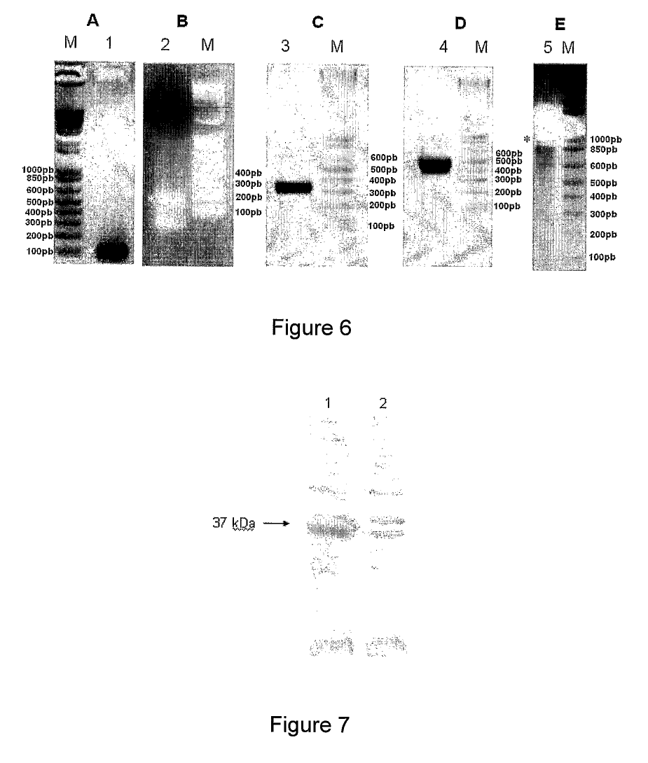

FIG. 8 shows a comparison of the strategy used for the oligonucleotides design in the method of the invention (A) in relation to the strategy used in other methods (B) described in the three patents cited in the background of invention.

### SEQUENCE LISTING

Sequence 1 represents the DNA sequence of a gene that codes the human Interferon β2 and is used as an example of synthesis of a 650 bp sequence.

Sequence 2 represents DNA sequence of a gene that codes for the human phosphodiesterase 5A enzyme and is used as an example of synthesis of a 970 bp sequence.

### DETAILED DESCRIPTION OF PREFERRED EMBODIMENTS

The present invention, aiming to overcome the problems found in current DNA synthesis techniques, provides a method that enables the creation of DNA molecules, entire genes, chromosomes and/or whole genomes based only on information available in databases or published in scientific literature, that is, without requiring the use templates from natural, preexistent DNA, or manipulated organisms or, yet, without requiring the use of biological materials such as clinical samples.

The DNA sequences assembled by the method of the invention keep fidelity to their original sequences. The method of the invention also enables codon optimizations. The present invention also enables the production of a DNA fragment with site-directed mutagenesis or the production of DNA fragments and/or genes with mutations such as deletions, insertions, substitutions etc., as well as simultaneous mutations, or any alterations on the target DNA sequences.

Additionally, the method of the invention does not require, in any step, any other enzyme besides a thermostable DNA polymerase. In the method of the invention the bacteria machinery is used to correct “nicks” generated when linking DNA sequences to the vector, so as to obtain chimeric DNA.

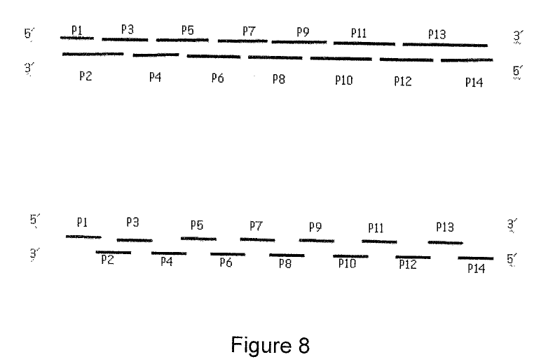

Although the synthesis of the initiator oligonucleotides is necessary, said oligos need not to be phosphorylated. The initiator oligonucleotides are designed in such a manner that there is just a small homologous region between the superior and inferior strands, without the need of synthesizing two entire strands, as shown in FIG. 8. These features of the method of the invention reduce considerably the cost of oligonucleotides synthesis.

The present invention has as another advantage the fact that at the end of the synthesis the target DNA sequence is practically definitive, that is, ready for use. There is no need to screen hundreds of clone sequences to find the correct construction, as required in other techniques.

Another significant advantage is that if the obtained target sequence presents any alteration, its sequence can be easily edited by using the synthesized oligonucleotides and the products of previous steps, therefore facilitating the correction of the mistake and avoiding the re-work of the whole process.

The method of the invention enables one to create DNA molecules, entire genes, chromosomes and genomes, and comprises the steps of:
(1) identification, in databases or scientific papers, of the desired DNA sequence to be synthesized;
(2) design of the initiator oligonucleotides by the user;
(3) synthesis of corresponding initiator oligonucleotides; and
(4) assembly of the desired nucleotide sequence(s).

In order to exemplify each step of the invention two situations are shown hereinbelow, although these examples are by no means intended to limit to scope of the invention:

1) a DNA sequence having up to 650 bases pairs (bp)—in this specific case a 638 bp sequence that code for human Interferon β2 was used; and

2) a DNA sequence with more than 650 bp—in this specific case a 970 bp sequence that code for the catalytic domain of human phosphodiesterase 5A was used.

These examples are further detailed below.

### Example 1

### Obtention of Sequences Having Up to 650 Base Pairs

First, the desired sequence to be assembled is selected. The initiator oligonucleotides are then manually designed and synthesized, in even numbers, corresponding to the double stranded DNA, in a continuous, successive and alternate manner. Using a DNA sequence of 638 bp, it could be divided in 14 shorter sequences, of about 50 bp each, wherein each of the 14 designed sequences should overlap its ends at least 10 bases over the adjacent ends, and so on, until all the chosen desired sequence is covered, as exemplified in Sequence 1. All oligonucleotide sequences were synthesized to a concentration of 0.01 nmol.

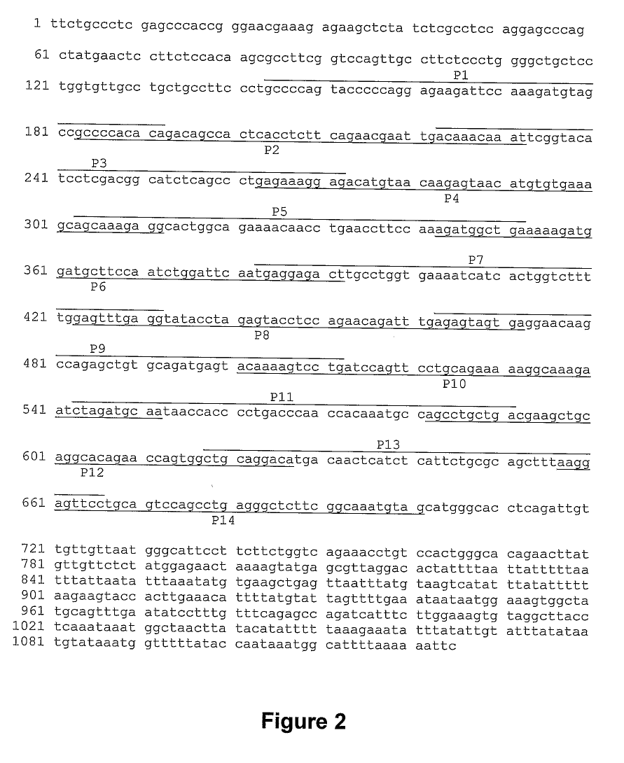

The DNA assembling method shown in FIG. 2 comprises the following steps:

(i) in (a) the oligonucleotides to be used are represented. The first step (b) involves the annealing of complementary regions of oligonucleotides P1 and P2 and its extension by DNA polymerase. The same procedure is performed with oligonucleotides P3 and P4, P5 and P6, P7 and P8, P9 and P10, P11 and P12, and P13 and P14, with the corresponding extensions in separated reactions. The amplified fragments ranging about 90 bp are purified from agarose gel using a commercial kit for DNA gel extraction;

(ii) the second step (c) consists of joining the obtained products in the previous step. As the oligonucleotide P2 contains the sequence of 3′ end complementary to the sequence of 3′ end of P3 (as well as P4 has to P5, P6 to P7, P8 to P9, P10 to P11, and P12 to P13) the P1+P2 product is annealed by homology to P3+P4 product and the oligonucleotides P1 and P4 are added in a PCR reaction to amplify the product (P1+P2+P3+P4). The same is performed in separated reactions to anneal P5+P6 and P7+P8 products by homology and amplify in presence of oligonucleotides P5 and P8, as well as to anneal the P9+P10 and P11+P12 products by homology and amplify in presence of oligonucleotides P9 and P12. In this step, the expected fragments have about 180 bp and are purified from agarose gel using a commercial kit for DNA gel extraction;

(iii) the third step (d) consists in the annealing of P1+P2+P3+P4 product by homology to P5+P6+P7+P8 product and in the amplification in the presence of oligonucleotides P1 and P8 forming the P1+P2+P3+P4+P5+P6+P7+P8 product. The P9+P10+P11+P12 product is annealed by homology to the P13+P14 product and amplified in presence of oligonucleotides P9 and P14, wherein, in this step, the expected fragments have about 300 bp and are purified from agarose gel using a commercial kit for DNA gel extraction, and

(iv) the fourth and last step (e) consists in the annealing of P1+P2+P3+P4+P5+P6+P7+P8 product by homology to P9+P10+P11+P12+P13+P14 product and in the amplification in the presence of oligonucleotides P1 and P14, wherein, in this step, the expected fragments have about 500 bp and are purified in gel. This amplification product results in the final target DNA (f).

The PCR reactions were performed under the following conditions:

The first 5 PCR amplification cycles generated the entire DNA products and are performed in an annealing temperature of 37° C. and the other cycles that amplified both strands are performed in an annealing temperature of 60° C. The PCR in the first step (b) was performed with 10 pmol of each oligonucleotide, using appropriate buffer for DNA polymerase, 2.5 units of thermostable DNA polymerase and 0.2 mM of each dNTP in a final volume of 50 μl (fifty microliters). 5 cycles were performed: i) denaturation for 45 seconds at 94° C., ii) annealing for 45 seconds at 37° C.; and iii) amplification for 1 minute at 72° C. Afterwards, 25 cycles were performed: i) denaturation for 45 seconds at 94° C., ii) annealing for 45 seconds at 60° C.; and iii) amplification for 1 minute at 72° C. In the posterior steps (c, d, e), the PCR reactions were performed in the same conditions, with the further addition of 2 μl of amplified and purified products (using a commercial kit for DNA gel extraction).

The PCR products were analyzed on 2% ultra pure agarose gel with 0.5 μg of Ethidium Bromide in Tris borate buffer (90 mM Tris/2 mM EDTA pH 8.0). A fragment with the appropriate size was cut from the gel, and the purified DNA using a commercial kit to purify DNA from the gel.

Finally, to clone the target sequence in the selected vector, initiator oligonucleotides can be synthesized, based at 5′ and 3′ ends of the target DNA sequence, containing sequences to restriction sites according to the vector that is being used for cloning. Alternatively, the sequences containing restriction sites (user's choice) can already be present at the initiator oligonucleotides, in case P1 and P14, situation where the initiators are so designed.

### Example 2

### Obtention of Sequences Having More than 650 Base Pairs

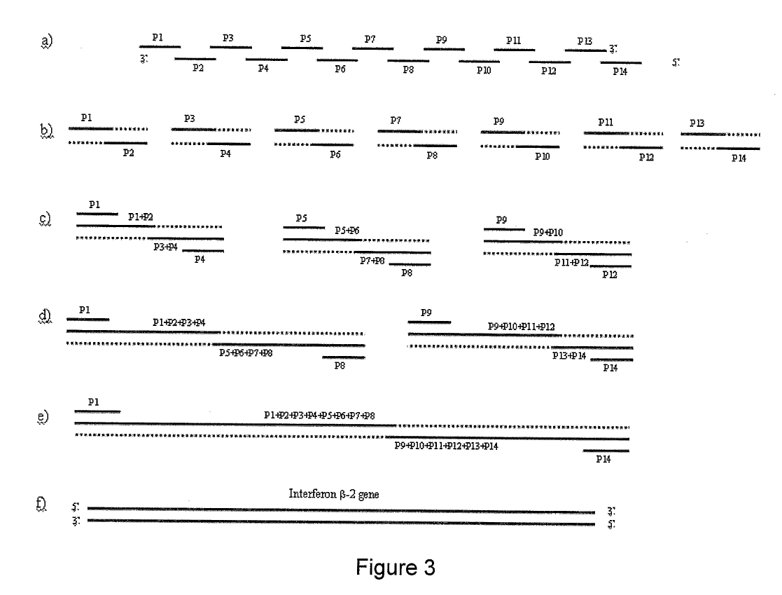

For DNA sequences having more than 650 base pairs, the designed oligonucleotides, as well as steps (i), (ii) and (iii), follow the same procedures shown in Example 1. The amplification of a nucleotide sequence with more than 650 base pairs is exemplified by the DNA sequence having 970 base pairs of Sequence 2. FIG. 3 shows the mechanism for amplification and assembly of a 970 base pairs sequence.

As in Example 1, the initiator oligonucleotides are demonstrated, as well as the procedures which in this case are more extensive than in Example 1.

(i) Step (b) involves annealing and amplification of the complementary regions of oligonucleotides P1 and P2. The same procedure is performed with oligonucleotides P3 and P4, P5 and P6, P7 and P8, P9 and P10, P11 and P12, P13 and P14, P15 and P16, P17 and P18, P19 and P20, P21 and P22, and P23 and P24, carried out on separated reactions. Afterwards, the amplified fragments with a size of approximately 90 bases pairs are purified using a commercial kit;

(ii) Step (c) consists in joining products obtained in the previous step. Here, as the sequence of the extremity 3′ end of oligonucleotide P2 is complementary to the sequence of 3′ of oligonucleotide P3 (as well as P4 is complementary to P5, P6 to P7, P8 to P9, P10 to P11 P12 to P13, P14 to P15, P16 to P17, P18 to P19, P20 to P21 and P22 to P23) the product P1+P2 is annealed by homology to product P3+P4 and the oligonucleotides P1 and P4 are added to perform the amplification of product (P1+P2+P3+P4). The products P5+P6 and P7+P8 are annealed by homology and amplified in the presence of oligonucleotides P5 and P8. The products P9+P10 and P11+P12 are annealed by homology and amplified in the presence of the oligonucleotides P9 and P12. The products P13+P14 and P15+P16 are annealed by homology and amplified in the presence of oligonucleotides P13 and P16 (product P13+P14+P15+P16). The products P17+P18 and P19+P20 are annealed by homology and amplified in the presence of oligonucleotides P17 and P20 (product P17+P18+P19+P20). The products P21+P22 and P23+P24 are annealed by homology and amplified in the presence of the oligonucleotides P21 and P24 (product P21+P22+P23+P24). In this step the expected fragments are of approximately 170 base pairs and they are purified from the gel using a commercial Kit;

(iii) the third step consists of annealing product P1+P2+P3+P4 by homology to product P5+P6+P7+P8 and amplification in presence of oligonucleotides P1 and P8 forming the product P1+P2+P3+P4+P5+P6+P7+P8. The product P9+P10+P11+P12 is annealed by homology to the product P13+P14+P15+P16 and amplified in the presence of oligonucleotides P9 and P16 forming the product P9+P10+P1+P12+P13+P14+P15+P16. The product P17+P18+P19+P20 is annealed by homology to the product P21+P22+P23+P24 and amplified in the presence of the oligonucleotides P17 and P24 forming the product P17+P18+P19+P20+P21+P22+P23+P24. On this step the expected fragments have approximately 330 base pairs and they are purified from the gel, using a commercial kit;

(iv) the fourth step consists of annealing the product P1+P2+P3+P4+P5+P6+P7+P8 by homology to product P9+P10+P11+P12 and amplification in the presence of the oligonucleotides P1 and P12 forming the product P1+P2+P3+P4+P5+P6+P7+P8+P9+P10+P1+P12. The product P13+P14+P15+P16 is annealed by homology to the product P17+P18+P19+P20+P21+P22+P23+P24 and amplified in the presence of oligonucleotides P13 and P24 forming the product P13+P14+P15+P16+P17+P18+P19+P20+P21+P22+P23+P24;

(v) the fifth step consists on the joining of the products P1+P2+P3+P4+P5+P6+P7+P8+P9+P10+P11+P12 and P13+P14+P15+P16+P17+P18+P19+P20+P21+P22+P23+P24 with the amplification of an intermediate product intended to increase the overlap area between the fragments. Therefore product P1+P2+P3+P4+P5+P6+P7+P8+P9+P10+P1+P12 and P9+P10+P11+P12+P13+P14+P15+P16 should be united, by homology annealing and amplified with oligonucleotides P1 and P16 forming the product P1+P2+P3+P4+P5+P6+P7+P8+P9+P10+P1+P12+P13+P14+P15+P16. In the same way, the products P9+10+P11+P12+P13+P14+P15+P16 and P13+P14+P15+P16+P17+P18+P19+P20+P21+P22+P23+P24 were annealed by homology and amplified with oligonucleotides P9 and P24 forming the product P9+P10+P11+P12+P13+P14+P15+P16+P17+P18+P19+P20+P21+P22+P23+P24. In this step the expected fragments have approximately 650 base pairs, which are purified in the gel (kit);

(vi) the sixth and last step consists on the annealing of the two products, P1+P2+P3+P4+P5+P6+P7+P8+P9+P10+P1+P12+P13+P14+P15+P16 and P9+10+P11+P12+P13+P14+P15+P16+P17+P18+P19+P20+P21+P22+P23+P24 by homology and amplification with oligonucleotides P1 and P24 in order to obtain product P1+P2+P3+P4+P5+P6+P7+P8+P9+P10+P19+P20+P21+P22+P23+P24, which is the desired target sequence with 970 base pairs.

Finally, to clone the target sequence in the chosen vector, initiator oligonucleotides can be synthesized, based on the 5′ and 3′ ends of the target DNA sequence, said oligonucleotides containing sequences for restriction sites according to the chosen vector for the cloning or the sequences for restriction siter can already be present in the initiators oligonucleotides, or yet the sequences for restriction site (user's choice) can already be present in the initiators oligonucleotides, in this case P1 and P24 in the occasion of initiator design by the user.

The assembly method disclosed on both examples above describes the use of at least 2 oligonucleotides for the first step of the assembly. It does not mean that more than 2 oligonucleotides could not be used for obtaining of the first assembly product; the conditions of the reactions can therefore be adjusted by the user.

The cloning of the synthetically obtained fragment can be performed in a vector for prokaryotic and/or eukaryotic cells. It is important to emphasize that, although the method of the invention may seem laborious until the final product assembly when extremely long sequences are to be obtained, ir provides several advantages in view of other known methods. The assembly and sequence of the used steps are easy to understand and they follow an irrefutable logical reasoning.

Experts will appreciate that other ways of performing the method of the present invention are possible from this description, and small modifications in the ways of performing the method herein described should be deemed as within the scope of the invention and of the following claims.

1. Method for the obtainment of chimeric nucleotide sequences comprising the steps of:
a) contacting at least two synthetic nucleotide sequences designed by the user, wherein at least part of said sequences comprises overlapping regions;
b) providing conditions so as said overlapping regions non-randomly bind to each other forming a double stranded region and at least one single stranded, non-overlapping region;
c) adding the corresponding building blocks at corresponding conditions so as a polymerase can perform an extension of said single stranded, non-overlapping regions into fully double stranded regions; and
d) non-randomly adding at least one additional synthetic nucleotide sequence designed by the user, wherein at least part of said additional sequence comprise at least one overlapping region with either end of the sequence obtained in step c) and non-randomly repeating steps b) and c) so as to obtain a non-random extended nucleotide target sequence in a step-by-step fashion.

2. Method, according to claim 1, wherein the fact that said polymerase is a thermostable DNA polymerase.

3. Method, according to claim 1, wherein the fact that said building blocks are deoxynucleotides.

4. Method, according to claim 1, wherein the fact that said building blocks are nucleotide/nucleoside analogs.

5. Method, according to claim 1, wherein the fact that at least one of said synthetic nucleotide sequences designed by the user comprises modified sequences in relation to the intended synthesized target sequences.

6. Method, according to claim 5, wherein said synthetic nucleotide sequence designed by the user comprises at least one restriction site deliberately designed by the user.

7. Method, according to claim 5, wherein said synthetic nucleotide sequence designed by the user comprises at least one sequence modification selected from the group consisting of insertions, deletions, inversions, substitutions, and combinations thereof.

8. Method, according to claim 1, wherein the synthesized target sequence is further linked to a cloning and or expression vector functional in prokaryotes or eukaryotes so as the said synthesized target sequence is replicated within the host organism thereby providing the large scale production thereof.

9. Method, according to claim 1, wherein the synthesized target sequence consists of entire genes, chromosomes, genomes and combinations thereof.

10. Chimeric nucleotide sequence being obtained by a method comprising the steps of:
a) contacting at least two synthetic nucleotide sequences designed by a user, wherein at least part of said sequences comprises overlapping regions;
b) providing conditions so as said overlapping regions non-randomly bind to each other forming a double stranded region and at least one single stranded, non-overlapping region;
c) adding corresponding building blocks at corresponding conditions so as a polymerase can perform an extension of said single stranded, non-overlapping regions into fully double stranded regions; and
d) non-randomly adding at least one additional synthetic nucleotide sequence designed by the user, wherein at least part of said additional sequence comprise at least one overlapping region with either end of the sequence obtained in step c) and non-randomly repeating steps b) and c) so as to obtain an non-random extended nucleotide sequence in a step-by-step fashion.
